(19) United States
(12) Patent Application Publication
(10) Pub. No.: US 2013/0078260 A1
(43) Pub. Date: Mar. 28, 2013

(54) COMPOUNDS TARGETING THE VEGF AND/OR HIF PATHWAY SUCH AS SORAFENIB OR VATALANIB FOR USE IN THE TREATMENT OF OTITIS MEDIA

(71) Applicants: Michael T. Cheeseman, Hempstead (GB); Steve D.M. Brown, Wantage (GB)

(72) Inventors: Michael T. Cheeseman, Hempstead (GB); Steve D.M. Brown, Wantage (GB)

(73) Assignee: MEDICAL RESEARCH COUNCIL, Swindon (GB)

(21) Appl. No.: 13/636,097

(22) PCT Filed: Mar. 18, 2011
(86) PCT No.: PCT/GB2011/000382

§ 371 Date: Dec. 3, 2012

(30) Foreign Application Priority Data
Mar. 22, 2010 (GB) 1004761.1

(51) Int. Cl. A61K 31/502; A61K 38/02; A61K 39/395; A61K 31/44; A61K 31/395

(52) U.S. Cl. CPC A61K 31/502; A61K 31/44; A61K 31/395; A61K 39/3955; A61K 38/02
USPC 424/158.1; 514/248; 514/346; 514/183; 514/8.1

(57) ABSTRACT

The present invention provides a compound which targets the VEGF and/or HIF pathways for use in the treatment and/or prevention of otitis media in a subject. The invention also provides a pharmaceutical composition comprising such a compound and a method for treating and/or preventing otitis media in a subject which comprises the step of administering such a compound or pharmaceutical composition to the subject.

### FIELD OF THE INVENTION

The present invention relates to a compound for use in the treatment and/or prevention of otitis media in a subject.

### BACKGROUND TO THE INVENTION

Otitis media (OM), inflammation of the middle ear (ME), is the most common cause of hearing impairment in children, potentially causing language delays, learning and behavioral disruption.

Acute OM (AOM) in infants and children is most often associated with bacterial infections involving Streptococcus pneumoniae, Haemophilus influenzae and Moraxella catarrhalis. About 40% of children have ME effusions 1 month after AOM and by 3 months after AOM, only about 10% still have ME effusion. This persistent effusive OM without the symptoms of infection is termed OM with effusion (OME). OME is very common in children 1-3 years old with a prevalence of 10-30% and a cumulative incidence of 80% by the age of four. Some children with OME will go on to develop chronic OM with complications such as tympanic membrane retraction pockets, erosion of the ossicular chain, cholesteatoma, chronic suppurative OM or otorrhea (tympanic membrane perforation and drainage of pus).

The high prevalence of the disease coupled with its recurrent and chronic nature accounts for the large number of tympanostomies (the insertion of ventilation tubes or ‘grommets’ in the tympanic membrane) undertaken in affected children. OM is still the most common cause of surgery in children in the developed world. Grommet insertion is the commonest operation in the UK (30,000 procedures per annum).

In clinical trials, decongestants, mucolytics, steroids, antihistamines and antibiotics have been found to be largely ineffective (Lous et al (2005) Cochrane Database Syst. Rev. CD001801). There is thus a clinical need for new medical treatments for OM and OME.

### DESCRIPTION OF THE FIGURES

FIG. 1. Hypoxia in the middle ear of Junbo mouse. Scale bars: A,B,C=50 μm; D=100 μm, G=20 μm; F=100 μm.

(A) Jbo/+ mouse labeled with 60 mg/kg Pimonidazole (PIMO) in vivo for 3 h, arrows indicate hypoxia in [ep] epithelium, [f] connective tissue fibrocyte, [mφ] macrophage; [tm] temporomandibular bone, [m] thickened inflamed mucosa, [ex] exudate contains a mixture of foamy macrophages (mφ) and polymorphonuclear cells (PMN). Immunohistochemistry with anti-PIMO antibody. Note * the cleft is an artifact produced by tissue processing.

(B) PIMO labeled WT mouse, the normal thin mucosa [m] is not stained.

(C) Unlabeled Jbo/+ mouse with OM is a negative control for anti-PIMO antibody.

(D) Hypoxia in foamy mφ.

(E) Time course of PIMO-labeling in Jbo/+ mice. The index scores a point for positive labeling cells in each of the following classes: inflammatory cells in the lumen, mucosal epithelium and mucosal connective tissues. Histogram bars are mean±SEM. 4 wk group size n=8, 7-8 wk n=10, 13-15 wk n=5, 31-37 wk n=7.

(F) Hypoxia in Eustachian tube epithelium [et] in a WT mouse is sharply demarcated from adjacent nasopharynx [np] epithelium.

(G) Ear exudate cytology from a Jbo/+ mouse: foamy mφ stained with rat anti-mouse F4/80 mAb and PMN.

FIG. 2. FACS analysis of Jbo/+ ear fluids. The middle ear WBC from a Pimonidazole labeled Jbo/+ mouse were stained with Ly6G and Ly6C (PMN marker), for PIMO (hypoxia), and Annexin V (apoptosis marker). The PMN population was gated on the Ly6G and Ly6C signal. Population (1) normoxic viable PMN, (2) hypoxic viable PMN, (3) hypoxic apoptotic PMN. Axis calibration is in log10 fluorescence.

FIG. 3. Western blotting for Hif-1α in Jbo/+ mice.

Lane 1 Jbo/+ bone marrow PMN.

Lane 2 Jbo/+ middle ear WBC show Hif-la positive bands.

The experiment was performed independently three times with the same result.

FIG. 4. Gene expression in middle ear of Jbo/+ mice. Real Time quantitative PCR in 4 wk and 10 wk pooled samples of Jbo/+ middle ear WBC and shown relative to those in 4 wk Jbo/+ blood WBC. Histogram bars are the mean fold-increase for triplicate assays (or Vegfa and Evil n=10). Error bars are the relative quantification (RQ) min and max and represent the SEM of the ΔCycle Thresholds.

FIG. 5. Treatment of Jbo/+ mice with PTK787/ZK 222584 reduces inflammatory changes in the middle ear. WT and Jbo/+ mice were treated with either 50 mg/kg or 75 mg/kg of drug for 4 wk, the sham control Jbo/+ groups received vehicle alone. The inflamed mucosa was thinner in the 50 mg/kg trial (a). In both trials drug treated groups had fewer blood (b) and lymphatic vessels (c) compared to their respective sham-treated controls; (d) the mucosal lymphatics were less dilated in 75 mg/kg-treated Jbo/+ mice than controls. In the 50 mg/kg trial, the WT group size was n=8; Jbo/+ drug n=15; Jbo/+ sham n=13. In the 75 mg/kg trial WT n=8; Jbo/+ drug n=15; Jbo/+ sham n=15. Histogram bars are mean ±s.e.m. 2-tailed Student t-tests.

FIG. 6. Treatment of Junbo mice with VEGF receptor inhibitors and the HSP90 inhibitor 17-DMAG moderates hearing loss. (A) change in Auditory Brain Stem response (ΔABR) in decibels (dB) in 15 d treatment with BAY 43-9006 (WT, sham, drug n=5, 11, 11 respectively); (B) ΔABR in 28 d treatment with SU-11248 (WT, sham, drug n=10, 15, 15); (C) ΔABR in 21 d treatment with PTK787

(WT, sham, drug n=9, 13, 15); (D) ΔABR in 28 d treatment with PTK787 (WT n=40, sham n=60, PTK787 n=30); (E) ΔABR in 28 d treatment with 17-DMAG (WT n=9, sham n=15, 17-DMAG n=15). In each experiment, the response to drug treatment was compared to the sham control. Histogram bars are mean±SEM. 1-tailed Mann Whitney U tests.

### SUMMARY OF ASPECTS OF THE INVENTION

Using mouse models of chronic OM, the present inventors have demonstrated that the inflamed middle ear in OM is a hypoxic environment and there is induction of Hypoxia Inducible Factor (HIF). They have also shown that inhibitors of Vascular

Endothelial Growth Factor (VEGF) receptors and HIF are able to reduce hearing loss and inflammatory changes in middle ear mucosa in such mouse models.

Targeting the HIF-VEGF signalling pathways offers an attractive therapeutic option in the prevention and/or treatment of OM.

Thus, in a first aspect, the present invention provides a compound which targets the VEGF and/or HIF pathways for use in the treatment and/or prevention of otitis media in a subject

In a first embodiment of this aspect of the invention the compound targets the interaction between VEGF and VEGFR.

In this embodiment, the compound may be a VEGF receptor inhibitor, such as vatalanib, sunitinib or sorafenib.

Alternatively the compound may be an anti-VEGF antibody or an anti-VEGF peptide.

In a second embodiment of this aspect of the invention the compound inhibits the HIF pathway.

In this embodiment, the compound may act by destabilising HIF. For example, the compound may inhibit the HIF chaperone HSP90. One example of such a compound is 17-DMAG.

In a second aspect, the present invention provides a pharmaceutical composition for use in the treatment and/or prevention of otitis media in a subject, which comprises a compound according to the first aspect of the invention.

Further aspects of the invention relate to:

(i) the use of a compound according to the first aspect of the invention in the manufacture of a pharmaceutical composition for the treatment and/or prevention of otitis media; and

(ii) a method for treating and/or preventing otitis media (OM) in a subject which comprises the step of administering to the subject a compound according to the first aspect of the invention, a pharmaceutical composition according to the second aspect of the invention, or a kit according to the third aspect of the invention.

### DETAILED DESCRIPTION

Otitis Media (OM)

Otitis media is inflammation of the middle ear, or middle ear infection. Otitis media occurs in the area between the ear drum and the inner ear, including a duct known as the eustachian tube.

Acute otitis media (AOM) is most often purely viral and self-limited, as is its usual accompanying viral URI (upper respiratory infection). There is congestion of the ears and perhaps mild discomfort and popping, but the symptoms resolve with the underlying URI. If the middle ear, which is normally sterile, becomes contaminated with bacteria, pus and pressure in the middle ear can result, and this is called acute bacterial otitis media. Viral acute otitis media can occasionally lead to bacterial otitis media in a very short time, especially in children. Bacterial cases may result in perforation of the ear drum, infection of the mastoid space (mastoiditis) and in very rare cases further spread to cause meningitis.

Otitis media with effusion (OME) is simply a collection of fluid that occurs within the middle ear space as a result of the negative pressure produced by altered Eustachian tube function. This can occur purely from a viral URI, with no pain or bacterial infection, or it can precede and/or follow acute bacterial otitis media. Fluid in the middle ear sometimes causes conductive hearing impairment, but only when it interferes with the normal vibration of the eardrum by sound waves. Over weeks and months, middle ear fluid can become very thick and glue-like (thus the name glue ear), which increases the likelihood of its causing conductive hearing impairment.

The term “otitis media” used herein relates to all forms of chronic OM, including OME, and recurrent acute OM caused by microbial infections, as opposed to acute OM arising as a result of a viral infection.

Chronic suppurative otitis media involves a perforation (hole) in the tympanic membrane and active bacterial infection within the middle ear space for several weeks or more. There may be enough pus that it drains to the outside of the ear (otorrhea), or the purulence may be minimal enough to only be seen on examination using a binocular microscope. This disease is much more common in persons with poor Eustachian tube function. Hearing impairment often accompanies this disease.

When the middle ear becomes acutely infected by bacteria, pressure builds up behind the ear drum. In severe or untreated cases, the tympanic membrane may rupture, allowing the pus in the middle ear space to drain into the ear canal. In a simple case of acute otitis media in an otherwise healthy person, the body's defenses are likely to resolve the infection and the ear drum nearly always heals. However, in some cases, drainage from the middle ear can become a chronic condition. As long as there is active middle ear infection, the eardrum will not heal.

Streptococcus pneumoniae and nontypable Haemophilus influenzae are the most common bacterial causes of otitis media. Tubal dysfunction leads to the ineffective clearing of bacteria from the middle ear.

Otitis media is usually diagnosed via visualization of the tympanic membrane in combination with the appropriate clinical history.

The term “otitis media” used in connection with the invention relates primarily to chronic OM, as opposed to acute OM arising as a result of a viral infection. The term may exclude OM arising as a result of Respiratory Syncytial Virus (RSV) infection.

VEGF/HIF Pathways

Mammalian VEGF ligands are 40 kDa glycoproteins which exist as several different splice variants and processed forms, including VEGF-A, -B, -C, -D, and -E.

VEGF ligands bind in an overlapping pattern to three receptor tyrosine kinases (RTKs) VEGFR-1, VEGFR-2 and VEGFR-3.

The binding of VEGFs to their receptors results in the formation of VEGFR homodimers and heterodimers. Dimerisation activates VEGFRs, leading to the autophosphorylation of intracellular tyrosine residues.

The term “VEGF pathway” includes targeting upstream regulators of VEGF gene or protein expression; or downstream effects of VEGF protein expression, for example signalling pathways that promote angiogenesis, vascular permeability of white blood cell recruitment.

The term “HIF pathway” includes targetting HIF-1α expression or activity. The HIF signalling pathway is upstream of VEGFR.

Duval et al ((2007) Mol. Biol. of the Cell 18:4659-4668) report another relevant HSP90 connection with VEGFR2: tyrosine phosphorylation of HSP90 by a client protein VEGFR2 is required for VEGFR2 signalling to endothelial nitric oxide synthase.

VEGFNEGFR Inhibitors

The VEGFNEGFR-targeting inhibitor may be a biological macromolecule, such as a peptide, antibody, or a DNA- or RNA-oligonucleotide, or it may be a small-molecule inhibitor.

As there are several VEGF family members whose epitopes are poorly conserved, it may be preferable to target VEGF receptors rather than the ligands themselves.

Anti-VEGF antibodies are known, such as the VEGF-specific humanised monoclonal antibody bevacizumab™. Bevacizumab™ directly inhibits the biological activity of VEGF by preventing the interaction of VEGF with VEGFR-1 and -2. Ranibizumab™ is another anti-VEGF humanised monoclonal antibody fragment which binds and inhibits VEGF-A. HuMV833™ is another anti-VEGF-A antibody currently under clinical trial.

Anti-VEGFR-2 antibodies include DC-101 and the mouse/human chimaeric monoclonal antibody IMC-IC11, together with the fully human anti-VEGFR antibodies IMC-2C6 and IMC-1121.

Aptamers are RNA- or DNA-oligonucleotides selected for their ability to bind proteins with high affinity and high specificity. Pegaptanib™ is an anti-VEGF RNA aptamer.

Alternatively the inhibitor may be a ribozyme which targets the VEGF/VEGFR pathway. Angiozyme, which was the first synthetic ribozyme to be tested as a therapeutic agent for human disease specifically cleaves VEGFR-1 RNA.

Another approach to target VEGF/VEGFR interactions involves the use of soluble receptors comprising the VEGF binding site to sequestrate VEGF and block VEGF signalling, such as VEGF-Trap™

It may also be possible to use VEGF splice variants to modulate the activity of VEGFRs.

The VEGFR inhibitor for use in the present invention may be selected from the following group: sorafenib, sunitinib, vatalanib, vandetanib, AZD-2171, SU-6668, CP-547632, pazopanib, BIBF-1120, Axitinib, AMG-706, AEE-788, EXEL-0999, EXEL-7647, XL-880, EXEL-2880, SU-14813, ZK-304709, E-7080, CHIR-265,

CHIR-258, OSI-930, BAY-579352, ABT-869, BMS-582664, KRN-951 and CEP-7055.

Further details of such small molecule inhibitors may be found in Kiselyov et al ((2007) Expert Opin. Invest. Drugs (2007) 16 (1):83-107).

The VEGF inhibitor may be of anilinophthalazine type, such as vatalanib; or urea type, such as sorafenib.

The VEGF inhibitor may be sorafenib, sunitinib or vatalanib.

HIF Pathways Inhibitors

A number of small molecule HIF-1 inhibitors are known including the following four compounds, which share the same thiophene oxadiazole core (marked in box):

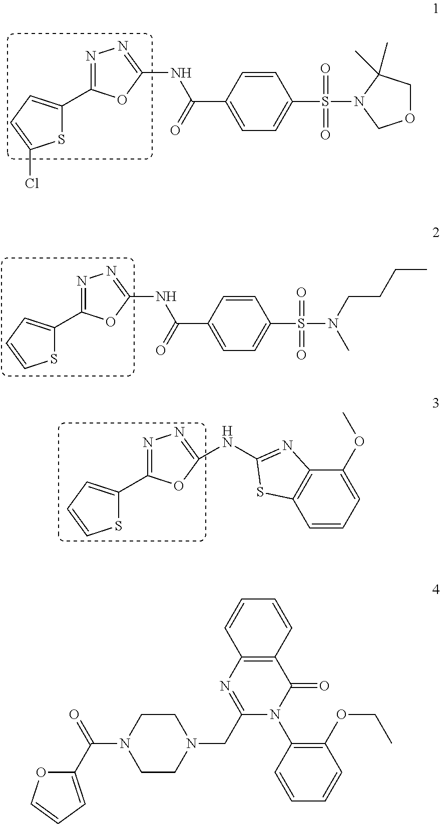 [SMILES: CC1(C)COCN1S(=O)(=O)c1ccc(C(=O)Nc2nnc(-c3ccc(Cl)s3)o2)cc1 ; CCCCN(C)S(=O)(=O)c1ccc(C(=O)Nc2nnc(-c3cccs3)o2)cc1 ; COc1cccc2sc(Nc3nnc(-c4cccs4)o3)nc12 ; CCOc1ccccc1-n1c(CN2CCN(C(=O)c3ccco3)CC2)nc2ccccc2c1=O]

Also under development as HIF-1 inhibitors are 2,2-dimethybenzopyran compounds.

The compound may act on HSP90, the chaperone to HIF. The compound may, for example, be 17-dimethylaminoethylamino-17-demethoxy-geldanamycin (17-DMAG). 17-DMAG induces HIF- 1 adestabilisation and degradation (van der Bilt et al (2007) Am. J. Pathol. 170:1379-1388; Milkiewicz et al (2007) J. Physiol. 583:753-766).

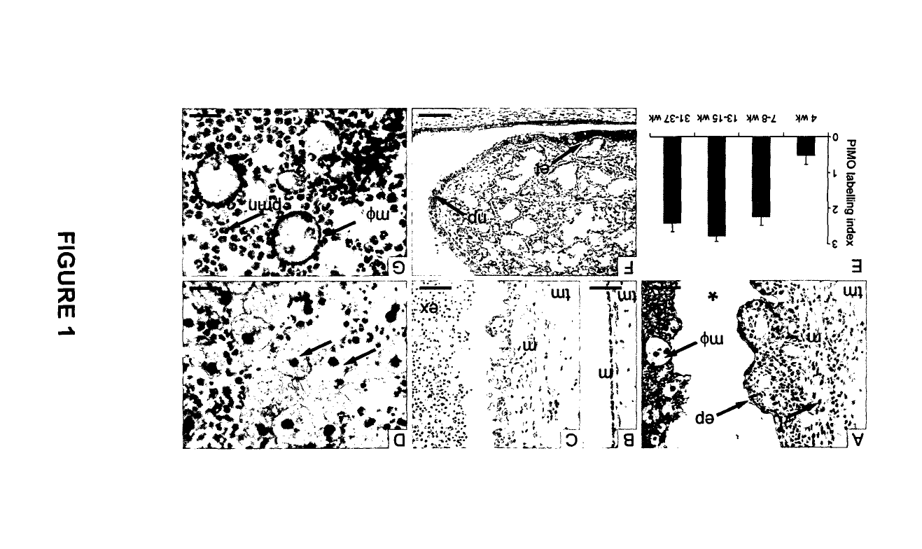

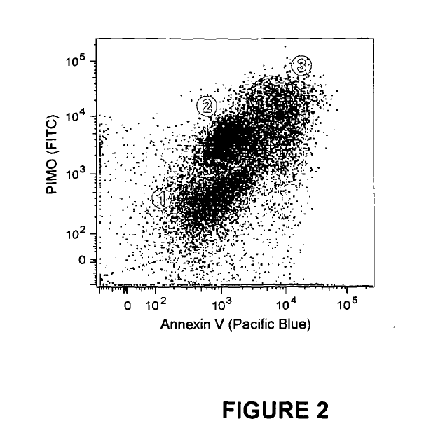

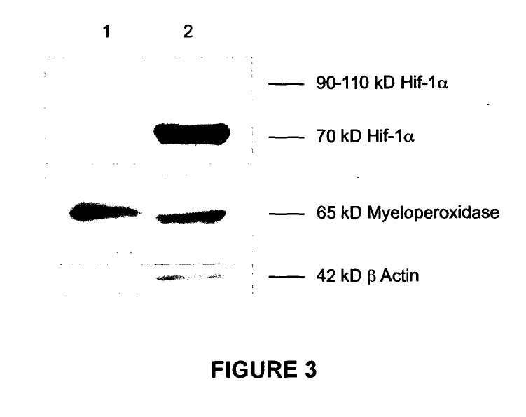

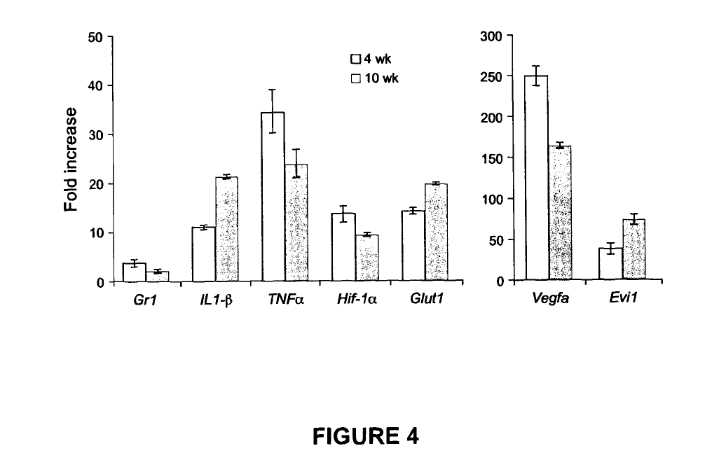

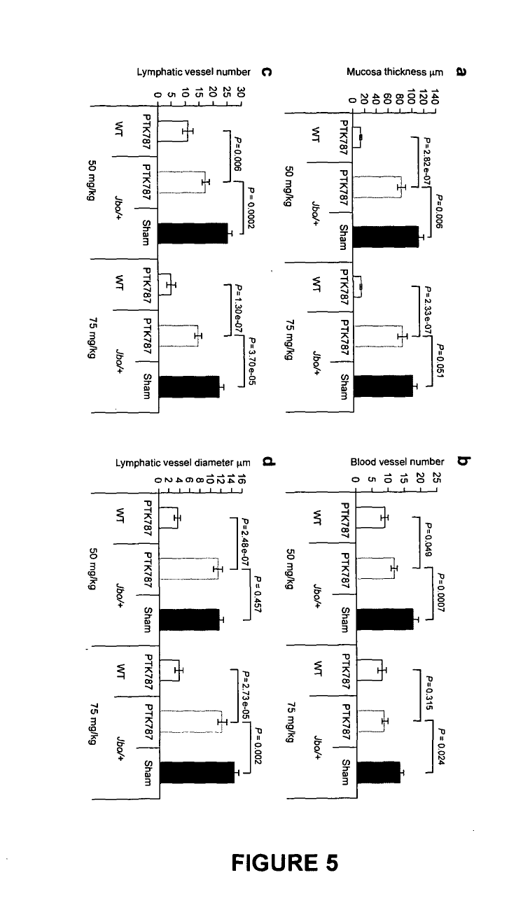

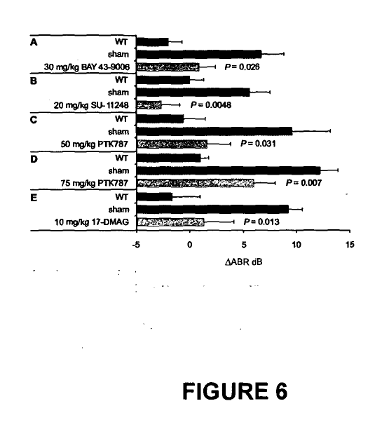

HSP90 inhibitors have been developed as anti-cancer drugs, for example, tanespimycin (17-AAG), Retaspimycin hydochloride (IPI-505), BIIB012, CNF2024, AUY922, STA-9090, IPI-493, SNX-5422 mesylate, BIIB028, KW-2478, AT13387, XL888, HSP990, MPC-3100, ABI-010; PU3, Radicicol and Novobiocin. See Trepel et al (2010) Nat. Rev. Cancer 10:537-549, Table 1; and Fukuyo et al (2010) Cancer Letts. 290:24-35 Figure 1).

Geldanamycin is a benzoquinone ansamycin antibiotic that manifests anti-cancer activity through the inhibition of HSP90-chaperone function. Related inhibitors include KOS-953, IPI-504 and IPI-493 (see Fukoyo et al, as above). Geldanamycin dissociates mature multi-chaperone complexes by inhibiting HSP90 ATPase activity and the released cleint proteins are subsequently degraded by the ubiquitin proteasome pathway. Gendalmycin induces the degradation of the HSP90 client protein HIF-1α.

Pharmaceutical Composition

In a second aspect, the present invention provides a pharmaceutical composition for use in the treatment and/or prevention of otitis media in a subject, which comprises a compound according to the first aspect of the invention.

The pharmaceutical composition may consist essentially of vatalanib. In other words, vatalanib may be the only ingredient of the composition which inhibits VEGFR. Vatalanib may be the only ingredient of the composition which is active in the treatment/prevention of otitis media. The pharmaceutical composition may substantially lack an ingredient for the treatment of RSV infection. The pharmaceutical composition may substantially lack any diindolylmethane-related indoles.

The pharmaceutical composition may also comprise a pharmaceutically acceptable carrier, diluent or excipient.

Subject

The compounds and compositions of the invention may be used to treat a subject having otitis media, for example a subject having chronic otitis media.

Alternatively the compounds and compositions of the invention may be used to treat a subject believed to be at risk from contracting otitis media. For example the subject may have suffered from OM in the past, or may have or be at risk from contracting a viral or bacterial infection associated with OM. The subject may have abnormal ear structure or physiology which pre-disposes then to OM, such as poor Eustachian tube function.

The subject may be a human subject, such as an adult or a child. In particular, the subject may be a child between the ages of 1 and 4 years.

The subject may be an animal subject, in particular a domestic or livestock animal such as a cat, dog, rabbit, guinea pig, rodent, horse, goat, sheep, cow or pig.

Administration

The compounds or pharmaceutical compositions of the invention may be administered by any route suitable for treating the middle ear.

Where appropriate, the pharmaceutical compositions may be administered by inhalation, in the form of a suppository or pessary, topically in the form of a lotion, solution, cream, ointment or dusting powder, by use of a skin patch, orally in the form of tablets, capsules or ovules. Alternatively the pharmaceutical compositions can be injected parenterally, for example intravenously, intramuscularly or subcutaneously.

The invention will now be further described by way of Examples, which are meant to serve to assist one of ordinary skill in the art in carrying out the invention and are not intended in any way to limit the scope of the invention.

### EXAMPLES

### Example 1

### Investigation of Hypoxia in Jbo/+ OM Mouse Model

In order to further characterize OM and to establish appropriate controls for gene expression studies in ME white blood cells (WBC), the WBC differentials in ear fluids and peripheral blood were examined. Jbo/+ mice did not have systemic leukocytosis (Table I). WBC counts in Jbo/+ ear fluids, 1.7-2.1×10⁶ per μl, were 1000 times greater than in blood and the cytology showed a high polymorphonuclear cell (PMN) count and smaller numbers of F4/80 positive foamy macrophages (mφ) (FIG. 1G).

TABLE I
White blood cells in blood and ears of Junbo mice
Supplemental Table I

| | | | | | percentage WBC differential | | | | |
|---|---|---|---|---|---|---|---|---|---|
| | | | n | WBC^{a} | neutrophils | lymphocytes | monocytes | eosinophils | basophils |
| 8 wk | blood | WT | 9 | 1.8 ± 0.1 | 33.3 ± 3.3 | 54.5 ± 1.2 | 3.9 ± 0.5 | 8.0 ± 3.1 | 0.1 ± 0.0 |
| | blood | Jbo/+ | 9 | 2.2 ± 0.3 | 33.1 ± 1.8 | 59.1 ± 2.2 | 2.9 ± 0.3 | 4.6 ± 1.0 | 0.1 ± 0.0 |
| | ear fluid | Jbo/+ | 8 | 1720 ± 409^{b} | ND^{c} | ND | ND | ND | ND |
| 15 wk | blood | WT | 12 | 2.8 ± 0.4 | 31.7 ± 4.5 | 62.9 ± 5.4 | 2.7 ± 0.9 | 2.2 ± 0.8 | 0.0 ± 0.0 |
| | blood | Jbo/+ | 51 | 1.9 ± 0.1 | 36.0 ± 1.8 | 55.6 ± 2.2 | 5.2 ± 0.6 | 2.9 ± 0.4 | 0.1 ± 0.0 |
| | ear fluid | Jbo/+ | 5 | 2094 ± 242^{d} | ND | ND | ND | ND | ND |

Mean ± SEM.
^{a}WBC × 10³ per μl. The mean WBC counts were significantly higher
in Jbo/+ ear fluids at 8 wk (^{b}P < 0.001) and at 15 wk (^{d}P < 0.001) compared with
Jbo/+ blood from same aged mice.
^{c}ND not done.

In inflamed tissues the physiological drivers of hypoxia are likely to be the uptake of oxygen by neutrophils and mtp coupled with their physical separation from an underlying vascular bed. This model is applicable to the accumulation of inflammatory cells within the 5-6 μl ME of the adult mouse. In order to evaluate hypoxia in the inflamed ME we injected mice in vivo with Pimonidazole (PIMO) a marker that labels tissues and cells with a pO₂<10 torr (˜1.5% O₂). Immunohistochemistry showed hypoxia in inflammatory cells within the ME lumen, the epithelium and in the connective tissues of the thickened, inflamed mucosa (FIG. 1A, 1C, 1D). Hypoxia was evident at 4 wk, increased at 7-8 wk and remained chronically elevated for >30 wk (FIG. 1E). In unaffected WT littermates with a normal air-filled bulla, the thin non-inflamed mucosa was not hypoxic (FIG. 1B). The only part of the ME apparatus that appeared hypoxic under normal physiological (non-inflamed) conditions was the Eustachian tube (FIG. 1F). FACS analysis provided additional evidence for hypoxia in PMN populations in ear fluids of Jbo/+ mice (FIG. 2). At two time points, 5-8 wk and 12-17 wk, there were similar percentages of viable and apoptotic cells. Older Jbo/+ mice had significantly greater populations of hypoxic apoptotic cells and correspondingly lower populations of normoxic apoptotic cells (Table I). Unlabeled Jbo/+ ME PMN and non-staining peripheral (normoxic) PMN from PIMO-labeled Jbo/+ mice served as negative controls. Propidium iodide staining showed that 7±2% of the Jbo/+ PMN population were necrotic. Although the ME fluids from Jf/+ mice were generally straw-colored serous effusions they contained populations of viable and apoptotic PMN that were hypoxic (Table II); 8±2% of the Jf/+ PMN population were necrotic.

TABLE II
Hypoxia in polymorphonuclear cell populations in ear fluids of
Pimonidazole-labeled Junbo and Jeff mice

| | | | PMN | | hypoxic PMN | | normoxic PMN | |
|---|---|---|---|---|---|---|---|---|
| | age | n | viable | apoptotic | viable | apoptotic | viable | apoptotic |
| Jbo/+ | 5-8 wk | 7 | 61 ± 4 | 21 ± 2 | 12 ± 4 | 49 ± 8 | 79 ± 5 | 45 ± 5^{b} |
| | 12-17 wk | 13 | 67 ± 5 | 15 ± 2 | 22 ± 6 | 85 ± 3^{a} | 63 ± 6 | 11 ± 2 |
| jf/+ | 7-11 wk | 12 | 52 ± 7 | 40 ± 8 | 45 ± 12 | 16 ± 2 | 47 ± 12 | 83 ± 2 |

Mean polymorphonuclear cell (PMN) percentages ± SEM.
Populations are expressed as percentages of their parent viable or apoptotic populations. The population of hypoxic apoptotic PMN was greater (^{a}P = 0.0024) in 12-17 wk Jbo/+ than in 5-8 wk Jbo/+ mice. The normoxic apoptotic PMN population was greater (^{b}P < 0.001) in 5-8 wk Jbo/+ than 12-17 wk Jbo/+ mice.

### Example 2

### Analysis of Hif-1α Protein Stabilization

Hypoxia stabilizes Hif1-α protein by inhibition of prolyl hydroxylase 2 (PHD2). Under normoxic conditions PHD2 hydroxylates prolyl residues in Hif1-α allowing Hif1-α to be bound and polyubiquitinated by the E3 ubiquitin ligase VHL and degraded by the proteasome. HIF is also regulated at transcriptional, translational and posttranslational levels by a range of inflammatory mediators that are linked through interaction between HIF and NF-κB, the transcriptional factor that is the master regulator of inflammation. A spectrum of inflammatory cytokines has been documented in the middle ears of patients with OM and in animal models of OM (Juhn, S. K. et al. (2008) Clin. Exp. Otorhinolaryngol. 1, 117-138). Among these, IL-1β and TNFα increase translation of HIF-1α mRNA (Frede, S. et al. (2007) Meth. Enzymol. 435, 405-419).

To confirm hypoxia was associated with Hif-1α protein stabilization, Western blots were performed which showed the presence of Mᵣ ˜90-110 kD and Mᵣ˜70 kD Hif-1α positive bands in Jbo/+ ear fluids but not in bone marrow PMN controls (FIG. 3). Gene and protein expression patterns were then investigated in ME of Jbo/+ mice. To establish suitable controls gene expression levels were first compared at 4 wk and 10 wk in WT and Jbo/+ blood. These were comparable (<2-fold differences) 4 wk Jbo/+ blood was then used as a reference point. The PMN marker Gr-1 was 2-4 fold higher in the ear fluids probably reflecting the higher PMN differential compared with blood (Table I; FIG. 1F) and any fold change over this limit was therefore interpreted as gene upregulation. There was higher Hif1-α(9-14 fold), Il-1β (11-21 fold) and Tnfa (24-34 fold) expression in Jbo/+ME fluid WBC compared to blood WBC (FIG. 4). II-β protein was detected in ear exudates (50,371±21,124 pg/ml n=7) but was below assay detection limits in all but 14% of serum samples from Jbo/+ and WT mice (n=22). Where IL-β was detectable in sera it ranged from 4-67 pg/ml. Tnfa was also elevated in ear exudates (12,321±1881 pg/ml (n=8) relative to serum (1.65±0.16 pg/ml (n=12) and 1.48±0.04 pg/ml (n=12) in Jbo/+ and WT respectively).

The downstream effects of Hif signaling in the Jbo/+ ME are evident in the upregulation of Glut1 (14-20 fold) and Vegfa (164-249 fold) (FIG. 4). Vegf protein was elevated >390-fold in ear fluids compared with serum, and these levels doubled from 4 wk to 8 wk (Table III). Importantly, Vegf was also elevated in Jf/+ ear fluids confirming that Hif signaling was not restricted to a single model of OM (Table III).

TABLE III
Vegf titers (pg/ml) in serum and ear fluids of Junbo and
Jeff mice Table III

| | | | Serum | | Ear |
|---|---|---|---|---|---|
| | | | WT | mutant | mutant |
| Junbo | 4 | wk | 29 ± 2 (9) | 30 ± 2 (9) | 11,665 ± 3,508 (8)^{a} |
| | 8 | wk | 32 ± 2 (11) | 31 ± 2 (14) | 23,653 ± 3,484 (11)^{bc} |
| Jeff | 7-11 | wk | 31 ± 1 (13) | 33 ± 1 (11) | 9,969 ± 2,403 (11)^{d} |

Mean ± SEM (n). The mean Vegf titer was higher in Jbo/+ ear fluids at 4 wk (^{a}P = 0.0035) and at 8 wk (^{b}P < 0.001) compared with in sera from their respective aged Jbo/+ mice. Mean Vegf titer was significantly higher at 8 wk than in 4 wk Jbo/+ ear fluids (^{c}P = 0.031). The mean Vegf titer was higher (^{d}P < 0.001) in Jf/+ ear fluids than sera.

The 38-74 fold increase of Evil expression in the ME of Jbo/+ mice (FIG. 4) is partly a reflection that it was barely detectable in blood (at cycle 35 compared to cycles 28-32 for the other genes). This result nevertheless raises the possibility that Evil perturbs hypoxia pathways via its role as a transcriptional factor. In Jbo/+ mice Evil expression may be driven by feedback loop(s) caused by mutant Evil protein dysregulating downstream hypoxic and inflammatory pathways. Evil might potentially impact on HIF signaling via Hif-1 α and Smad3 that coactivate Vegf expression in mouse macrophages. Alternatively, interactions may occur via the distal zinc-finger domain of Evil which raise AP-1. A mutation in Evil might affect Vegf expression as AP-1 has binding sites within the human VEGF promoter. In the case of the Jeff mutant, there are no obvious connections between Fbxol1 and HIF signaling and this argues that dysregulated HIF signaling may also be a downstream event in chronic OM.

### Example 3

### The Effect of Inhibition of VEGFR Signalling and Hsp90 in OM Mouse Models

Vegf acts to induce angiogenesis, increases vascular permeability and recruitment of WBC and may therefore contribute to OM pathogenesis by causing conductive hearing loss and secondary cochlear dysfunction via inflammatory mediators and toxins diffusing through the round window.

To test the hypothesis that Vegf has a pro-inflammatory role, Junbo mice were treated with small molecule VEGFR inhibitors. The Junbo mouse is the better mouse model for therapeutic trials because the OM phenotype is more highly penetrant. For instance at the time of dissection, serous or yellow fluid was clearly visible behind one (57%) or both (21%) eardrums in 7-11 wk Jf/+ mice (n=14) whereas fluid was visible behind one (14%) or both (79%) eardrums in 8 wk Jbo/+mice (n=54) (data pooled from sham control groups). It is noteworthy that even when no fluid was grossly evident, Jbo/+ mice had some degree of microscopic OM.

In Jbo/+ mice there was progressive hearing loss from 4 wk onwards. In independent trials, treatment of Jbo/+ mice for 2 wk with BAY 43-9006, 3 wk with 50 mg/kg PTK787/ZK 222584 (hereafter referred to as PTK787) or 4 wk with 75 mg/kg PTK787 reduced hearing loss (Table IV and FIG. 6). The trial with BAY 43-9006 was terminated after 2 wk when mice suddenly became piloerect. Although BAY 43-9006 was not as well tolerated as PTK787, the positive therapeutic response to treatment is compelling evidence for the importance of VEGFR as a HIF-pathway target for OM treatment.

TABLE IV
Change in Auditory Brain Stem (ΔABR) response in decibels (dB) in
the Junbo mouse treated with vascular endothelial growth factor receptors
(VEGFR)

| | | | ΔABR (dB) | | |
|---|---|---|---|---|---|
| VEGFR inhibitor | dosage | days | WT | Jbo/+ drug | Jbo/+ sham |
| PTK787/ZK 22584 | 50 mg/kg | 21 | −0.6 ± 1.1 (8) | 1.7 ± 2.1 (15)^{a} | 9.6 ± 3.6 (13 )^{b} |
| | 75 mg/kg | 28 | 0.0 ± 0.9 (8) | 6.3 ± 3.3 (8)^{c} | 15.0 ± 2.8 (13)^{d} |
| BAY 43-9006 | 30 mg/kg | 15 | −2.0 ± 1.2 (5) | 0.9 ± 1.5 (11)^{e} | 6.8 ± 2.1 (11)^{f} |

Moderation of hearing loss was accompanied by reduced inflammatory change in the ME mucosa. Although PTK787/ZK 222584 -treated Jbo/+ mice had fluid and inflammatory cells in the bulla lumen, treatment moderated mucosal thickening in the 50 mg/kg trial (FIG. 5a) and in both trials angiogenesis and lymphatic vessel number were reduced (FIG. 5b & c). Lymphatic vessel dilation was only moderated in the 75 mg/kg dosage group (FIG. 5d). In the 2 wk trial with BAY 43-9006, there was reduction in lymphatic vessel number (10.8±1.1 n=11 mice versus 16.4±1.0 n=11, P=0.0012) and vessel dilation (9.9±1.0 μm n=11 versus 14.2±0.9 μm n=11, P=0.0072) but not in mucosal thickening or angiogenesis, perhaps because neither were sufficiently advanced after 2 wk. The inventors went on to test the HSP90 inhibitor 17-DMAG which acts to destabilize HIF-la and its use also moderated hearing loss (FIG. 6).

In contrast to the positive therapeutic response to small molecule VEGFR inhibitors, a PPARγ agonist Rosiglitazone did not moderate hearing loss in Jbo/+ mice (Table V). We included Rosiglitazone in our trial because it has been shown to modulate reactive oxygen species generation and upstream modulators of HIF signaling, NFKB and Hif-1α in allergic airway disease in mice (Lee et al., 2006).

TABLE V
Change in Auditory Brain Stem (ΔABR) response in decibels (dB) in
Junbo mice treated with the PPARγ agonist Rosiglitazone

| | | | | |
|---|---|---|---|---|
| | | ΔABR | | |
| | | (dB) | | |
| dosage | days | WT | Jbo/+ drug | Jbo/+ sham |

| | | | | | |
|---|---|---|---|---|---|
| Rosiglitazone | 20 mg/kg | 28 | 1.4 ± | 9.7 ± | 13.7 ± |
| | | | 1.4 (8) | 3.3 (15)^{a} | 3.0 (15)^{bc} |

Mean ΔABR (dB) ± SEM (n).
The mean ΔABR was significantly higher in both drug (^{a}P = 0.041) and sham-treated Jbo/+ mice (^{b}P = 0.002) compared to WT mice whereas Jbo/+ drug and sham-treated groups were not significantly different (^{c}P > 0.05).

In conclusion, ME cellular hypoxia may be a common feature of chronic OM that is not revealed by physical measurements of oxygen in ME gases or fluids.

It has been shown that the ME in the Junbo model of OM is characterized by chronic inflammatory hypoxia, raising the possibility that inflammatory cytokines such as TNFα and IL-β play a role in modulating Hif1-α. Hif1-α signaling is associated with upregulation of Vegf. The key findings of ME hypoxia and upregulation of Vefg were independently confirmed in Jeff, a second mouse model of chronic OM.

The finding that VEGFR inhibitors PTK787and BAY 43-9006 reduce the progression of hearing loss supported the hypothesis that Vegf plays an important pro-inflammatory role in chronic OM.

Taken together, this work indicates that OM resembles other diseases of chronic hypoxic inflammation such as rheumatoid arthritis (Grosios, K. et al. (2004) Inflamm. Res. 53, 133-142) and opens up new avenues of research to target HIF and VEGF pathways in the treatment of chronic OM.

Materials and methods

Mice. The humane care and use of congenic C3H/HeH Junbo (Parkinson, N., et al. (2006) PloS Genet. 2, e149) and Jeff mice on a mixed C3H/HeH and C57BL/6J genetic background (Hardisty-Hughes, R. E., et al. (2006) Hum. Mol. Genet. 15, 3273-3279.) was under the appropriate UK Home Office license.

Sample collection. Blood was collected from the retro-orbital sinus of mice under terminal anesthesia induced by an i.p. overdose of sodium pentobarbital. Measured volumes (0.5-2 μl) of ear fluids from each mouse were collected into ice cold PBS.

Pimonidazole labeling. Mice were labeled 3 h in vivo by i.p. injection with 60 mg/kg Pimonidazole (PIMO) (Hypoxyprobe, HPI Inc) dissolved in 100 μl of sterile PBS. The head was fixed for 48 h in 10% neutral buffered formalin then decalcified with Formical (Decal Corp) for 72 h. Wax embedded 3 μm dorsal plane sections of the ME were immunostained for PIMO. For FACS, ear fluid samples were stained with anti-PIMO FITC, anti-mouse Ly6G and Ly6C PerCP-Cy5.5 (BD Pharminogen) and anti-Annexin V Biotin (BD Pharminogen) /Streptavidin Pacific Blue (Invitrogen). Propidium iodide (BD Pharminogen) was used to assess necrotic cells. 50 μl EDTA blood samples were diluted in 100 μl FACS buffer then treated with RBC lysis buffer (BD Pharminogen).

HIF Western blotting. Jbo/+ bone marrow PMN were isolated on a 52/64/72% discontinuous isotonic Percoll gradient (Sigma-Aldrich) and ME fluid from 5 mice was collected in 150 μl aliquots of ice-cold lysis buffer (Ambion) with protease inhibitor (Sigma-Aldrich). 35 μg protein samples of combined cytoplasmic and nuclear protein extract and mol wt markers were subjected to SDS-PAGE on 7.5% Tris-HCl gels. Nitrocellulose blots were blocked and incubated overnight at 4° C. with primary antibodies: 1:500 rabbit anti-HIF-1α (Novus Biologicals); 1:1000 goat IgG anti-human/mouse myeloperoxidase, (R&D Systems); 1:500 rabbit anti-βActin (Abeam). HRP-conjugated goat anti-rabbit IgG (Abcam) or HRP rabbit anti-goat IgG (Abeam) secondary antibodies were used at 1:2000 for 1 h and with a chemiluminescent detection system (Pierce Supersignal West Pico).

Real time quantitative PCR (RT-qPCR) and analysis. Total RNA from whole blood or ME fluids (collected on dry ice) was isolated from WT or Jbo/+ (5 male, 5 female mice) using a Mouse RiboPure kit (Ambion). RNA quantity was measured on a Nanodrop 8000 (Thermo Fisher Scientific) and the integrity assessed by gel electrophoresis before pooling equal quantities of RNA. Double stranded cDNA was synthesized from 1 pg of total RNA using High Capacity cDNA archive kit (Ambion). RT-qPCR was performed using TaqMan® Gene Expression Assays (Table VI), using TaqMan® Fast Universal PCR Master Mix on a 7500 Fast Real-Time PCR System (Applied Biosystems).

TABLE VI
RT-qPCR assays

| gene | TaqMan ® assays |
|---|---|
| Evi1 | Mm00514814_m1 |
| Glut1 | Mm00441473_m1 |
| Gr1 | Mm00833903_m1 |
| Hif-1α | Mm00468869_m1 |
| IL-1β | Mm01336189_m1 |
| TNF-α | Mm00443258_m1 |
| Ppia | Mm02342429_g1 |
| Vegfa | Mm00437304_m1 |

Vegf, IL-1β, TNFα protein assays. Blood was collected into serum-gel clotting activator tubes (Sarstedt). ME fluid samples in PBS were centrifuged at 5000 g for 20 s at 8° C. Supernatants and serum samples were stored at −80° C. until assay. Vegf was measured using a Quantikine mouse Vegf ELISA kit (R&D Systems) and IL-β and TNFα with a Fluorokine multianalyte profile (MAP) kit (R&D Systems).

Drug treatment, ABR and analysis of inflammation. 27-29 d old WT and Jbo/+ mice were dosed by oral gavage once a day for 2 wk with BAY 43-9006, 4 wk with 50 mg/kg PTK787, 4 wk with 75 mg/kg PTK787, 4 wk with Rosiglitazone or with 10 mg/kg 17-dimethylaminoethylamino-17-demethoxy-geldanamycin (17-DMAG). Stock solutions of aqueous PTK787 (LC Laboratories), or DMSO stock solutions of BAY 43-9006 (LC Laboratories) and Rosiglitazone (Molekula) were frozen at −20° C. then diluted 10-fold in 2% methyl cellulose for administration. The sham

Jbo/+ group was matched for age, gender and pre-trial ABR (30-60 dB) and received vehicle alone. A click-evoked ABR (Zheng, Q. Y., et al. (1999) Hearing Res. 130, 94-107) was measured at the beginning and end of the trial (or after 3 wk in the 50 mg/kg PTK787 trial). For ABR with recovery, anesthesia was induced by i.p. injection with a mixture of 10 mg/kg xylazine and 100 mg/kg ketamine and was reversed by 5 mg/kg atipamezole hydrochloride. Histology was assessed at 4 wk in both PTK787 trials.

Image capture and analysis. Digital images were captured on an Olympus BX51 microscope using ×20, ×40 or ×60 Plan Achromat objectives with neutral density filter, on a ColorView Soft Imaging System software using automatic exposures. In the drug trials, H&E stained sections for image analysis were scanned at ×400 magnification on a Nanozoomer digital pathology system (Hamamatsu Photonics). Eardrum thickness and the thickness of the mucosa along its medial surface (avoiding the cochlea and the region adjacent to the opening of the Eustachian tube) were averaged from 5 measurements. The number of capillaries and lymphatic vessels and their respective diameters were measured in a standardized 1000 μm length of medial mucosa. The treatment and genotype were blinded from the assessor when making the morphological measurements.

Statistics. Student t-tests or Mann Whitney U tests (for ABR measurements where interval data was in 5 dB increments) were performed and test values p<0.05 were considered significant. Percentages were arcsine transformed before using t-tests. In the drug trials, 1-tailed tests were used compare drug and sham-treated groups, otherwise 2-tailed tests were used.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1-12. (canceled)

13. A method for treating and/or preventing otitis media (OM) in a subject which comprises the step of administering to the subject a compound which targets the VEGF pathway and/or HIF pathway.

14. The method according to claim 12, wherein the compound targets the interaction between VEGF and VEGFR.

15. The method according to claim 12, wherein the compound is a VEGF receptor inhibitor.

16. The method according to claim 12, wherein the compound is vatalanib or sorafenib.

17. The method according to claim 12, wherein the compound is valalanib.

18. The method according to claim 12, wherein the compound is an anti-VEGF antibody.

19. The method according to claim 12, wherein the compound is an anti-VEGF peptide.

20. The method according to claim 12, wherein the compound destabilises HIF

21. The method according to claim 12, wherein the compound inhibits HSP90.

22. The method according to claim 12, wherein the compound is 17-dimethylaminoethylamino-17-demethoxy-geldanamycin (17-DMAG).

23. The method according to claim 12, wherein the compound is administered as a composition comprising the compound and a pharmaceutically acceptable carrier, diluent, or excipient.
